(19) United States
(12) Patent Application Publication
(10) Pub. No.: US 2022/0241493 A1
(43) Pub. Date: Aug. 4, 2022

(54) CRADLE FOR A MEDICAL DEVICE

(71) Applicant: Roche Diabetes Care, Inc., Indianapolis, IN (US)

(72) Inventors: Ahmet Konya, Mannheim (DE); Roland Hans Wiegand, Mannheim (DE)

(21) Appl. No.: 17/722,172

(22) Filed: Apr. 15, 2022

Related U.S. Application Data
(63) Continuation of application No. PCT/EP2020/078821, filed on Oct. 14, 2020.

(30) Foreign Application Priority Data
Oct. 16, 2019 (EP) 19 203 682.0

(51) Int. Cl. A61M 5/14; A61M 5/142

(52) U.S. Cl. CPC A61M 5/1415; A61M 5/14248; A61M 2230/201; A61M 2005/1416; A61M 5/1413

(57) ABSTRACT

Disclosed herein is a cradle for a medical device. The cradle has an inner cradle side and an opposing outer cradle side, wherein the outer cradle side is adapted for attachment to a patient's skin with at least part of the outer cradle side facing the patient's skin. The medical device has a medical device coupler, wherein the medical device coupler is adapted for coupling the medical device to the cradle such that part of the medical device is facing the inner cradle side. The cradle further includes a drain fluidly coupling the inner cradle side with the outer cradle side.

### RELATED APPLICATIONS

This application is a continuation of PCT/EP2020/078821, filed Oct. 14, 2020, which claims priority to EP 19 203 682.0, filed Oct. 16, 2019, the entire disclosures of both of which are hereby incorporated herein by reference.

### BACKGROUND

This disclosure relates to cradles that are used for temporarily coupling a medical device to a patient's body. This disclosure further relates to medical systems that include a cradle and a medical device. This disclosure further relates to a method of coupling a medical device to a patient's skin via a cradle. This disclosure also relates to a method of venting a space between a cradle and a medical device.

Medical devices that are directly attached to a patient's body for a continuous application time period of typically a number of days up to a number weeks or even months are used in increasing numbers. For example, some insulin pumps that are available for the therapy of diabetes mellitus are designed to be adhesively attached to a patient's skin and provide a continuous insulin supply according to a basal infusion schedule as well as insulin boli on demand. Similarly, continuous glucose measurement devices are used in combination with an insulin infusion pump or separately in order to provide substantially continuous glucose measurements, typically using an electrochemical sensor. Such medical devices that are designed for skin attachment are also referred to as patch devices. In dependence of the overall design and system architecture, they may either be directly attached to a patient's skin by way of an adhesive layer at a proximal or bottom side of the device, or may be indirectly attached via a cradle that is arranged between a patient's skin and the medical device. Typically, the cradle carries skin-contacting or skin-piercing elements such as an infusion cannula and/or a transcutaneous sensor element that project from the bottom side, particularly, the proximal side of the cradle. Once the cradle is attached, it has a fixed position relative to the skin and therefore provides a platform for the subsequent coupling of the medical device and skin-piercing elements. The cradle may stay attached to a patient's skin for the whole application time, while the medical device as such may be attached to and temporarily removed from the cradle as desired.

The separate provision of a cradle and the medical device as explained before, however, is critical regarding the presence of humidity and liquid, such as water. Typically, some gaps or generally space are/is present between the medical device respectively its housing and the cradle. In such gaps or space liquid may be present, e.g., resulting from water contact when swimming or showering. Typically, such water further carries a substantive amount of dust, dirt, microbes, germs and the like which might cause skin irritation or are critical in view of the proximity of one or more skin-piercing element(s). The situation is particularly critical in the typical case of narrow gaps between the cradle and the medical device housing, since resulting capillary effect tends to hold the liquid in place. To safely prevent the potential problems that may result from the long-term presence of humidity and liquids as explained before, constructive measures must be taken and/or the cradle and the medical device must be separated and dried by a patient after the exposure to water, which however is cumbersome and often omitted.

EP 3 251 585 A1 discloses a body-mountable device, in particular a patch for a medical assembly, comprising a housing portion adapted to receive the medical assembly and having a bottom side, and further comprising a fastening means which is connected to the housing portion and adapted to affix the housing portion to the body of a patient, such that the bottom side is facing the body, wherein a fluid-guiding component is configured on the bottom side of the housing portion for guiding a fluid emanating from the body to the periphery of the housing portion. The allows removal of humidity and liquids, such as sweat under the housing portion, but does not address the problem of humidity or liquid between the housing portion (corresponding to a cradle) and the medical device. To the contrary, the housing portion of the cradle has a wall at its bottom side that is impermeable for the fluid. It does accordingly not allow the removal of liquid from an area between the medical assembly and the housing portion respectively cradle.

### SUMMARY

This disclosure improves the state of the art regarding design of cradles and medical systems having a cradle and a medical device.

In use, i.e., in a state in which the cradle is attached to a patient's skin, a medical device is attached to the patient's skin via the cradle. By means of a draining structure (also referred to herein as a “drain”), water, humidity or the like, which might accumulate between the medical device and the inner cradle side, can be guided to an exterior of the cradle. The draining structure helps to vent a space between the medical device and the inner cradle side, and thus helps to reduce the potential problems that may otherwise result from the long-term presence of humidity, contaminations, and liquids as explained before.

In an embodiment, the draining structure comprises at least one through hole fluidically (“fluidically” and “fluidly” are used interchangeably herein) coupling the inner cradle side with the outer cradle side. The through hole forms a passage through the material of the cradle, such that water, humidity or air can transit the cradle from the inner cradle side to the outer cradle side, and vice versa. The draining structure may comprise a plurality of through holes, which are spread over the cradle. A part or all of the plurality of through holes may be fluidically coupled to each other by one or more grooves, channels or guides.

According to a further aspect, a medical system is disclosed which includes a cradle according to any embodiment as described above and/or further below. The medical system further includes a medical device. The medical device includes a cradle coupling structure, the cradle coupling structure being complementary to the medical device coupling structure of the cradle. The medical device further includes a proximal medical device side, wherein in a configuration in which the medical device is coupled to the cradle, the proximal medical device side faces the inner cradle side.

The directional expressions “proximal” and “distal” are referred to with respect to the skin of the patient's body in a configuration of use. “Proximal” refers to a direction pointing towards the patient's skin while “distal” refers to a direction pointing way from the patient's skin. The expression “periphery” refers to a lateral boundary traverse to a direction as defined by “proximal” and “distal.” The expression “plurality” refers to any natural number greater than one, i.e., 2, 3, 4 and so on.

In an embodiment the cradle includes a cradle base and a wall, wherein the wall projects away from the cradle base and comprises two opposing wall sides. One of the two opposing wall sides is at least partly formed by at least part of the outer cradle side, while the other of the two opposing wall sides is at least partly formed by at least part of the inner cradle side. The two opposing wall sides, which are at least partly formed by the at least part of the outer cradle side and the at least part of the inner cradle side, respectively, are fluidically coupled to each other by the drain. Accordingly, water, humidity, and/or air may transit the wall of the cradle by means of the draining structure. In use, part of the cradle base can be used to attach the cradle to a patient's body, such that part of the cradle base faces the patient's skin. Since the wall projects from the cradle base a draining structure located in or at the wall is usually not covered by a patient's skin. Therefore, a fluid flowing through the draining structure is less likely hindered by a patient's skin.

For example, the at least one through hole of the draining structure can be located at a free end of the wall, i.e., the end of the wall opposite to the end of the wall that is connected to the cradle base. Preferably, the wall and the cradle base form a one-piece entity, such that the wall and the cradle base cannot be separated without damaging the wall and/or the cradle. The cradle base and the wall can be made from a single material.

The at least one through hole of the draining structure can form an interruption of the wall, i.e., the at least one through hole extends from the free end of the wall to the end of the wall, which is connected to the cradle base. The wall is thus formed by a plurality of wall elements, which are spaced apart along the circumference of the cradle base. A draining structure which comprises an interruption of the wall helps to vent a space between the medical device and the wall of the cradle. In such embodiment, the at least one through hole may in particular be formed by a slit that extends in the wall from the cradle base to the free end of the wall. The slit may has a length in a direction projecting away from the cradle base that is larger than the slit width, i.e., the distance between two adjacent wall elements separated by the respective slit.

In an embodiment, the slit has a width which is equal to or less than half of the circumferential length of the cradle. In particular, the slit can have a width which is at least 30% of the circumferential length of the cradle. Accordingly, two wall elements may be displaced to each other in a direction along the circumference of the cradle of more than 30% and less or equal to 50% of the circumferential length of the cradle.

In an embodiment, one or more wall elements have a length in the circumferential direction of the cradle which is smaller than 50% of the circumferential length of the cradle and/or more or equal than 5% of the circumferential length of the cradle.

The wall may consist of at least two wall elements; preferably the wall consists of at least three wall elements, wherein two neighboring wall elements are separated from each other in the circumferential direction of the cradle by a through hole.

In an embodiment, the at least one through hole of the draining structure is located at the transition of the wall and the cradle base, thereby extending from the wall into the cradle base. Accordingly, the draining structure permits draining of a space between the medical device and the cradle base, as well as a space between the medical device and the wall of the cradle. In addition, the at least one through hole located at the transition of the wall and the cradle base may further extend to the free end of the wall. Alternatively, the through hole may not extend to the free end of the wall, thereby having a boundary at the free end of the wall. The boundary or stay, respectively, helps to increase a deformation resistance of the wall and/or the cradle, when compared to embodiments having a through hole, which extends from the free end of the wall to the cradle base.

In an embodiment, the wall forms a border strip extending over at least half of the circumferential length of the cradle base. The border strip thus at least partially surrounds the cradle base. In another embodiment the border strip extends over the entire circumferential length of the cradle base.

Typically, but not necessarily, the wall has a constant or substantially constant height as measured from the cradle base to the free end of the wall, i.e., in the distal direction. Further typically but not necessarily, the wall may have a constant or substantially constant thickness traverse to the proximal direction or distal direction, respectively.

The circumferential wall and the cradle base may be formed integrally, e.g., from plastic material. The medical device coupling structure may be fully or partly formed integrally with the wall.

In a coupled state of the medical device and the cradle, the medical device can be fully or partly received in the interior that is circumferentially delimited by a circumferential wall, such that a periphery of the medical device, particularly a housing of the medical device at least partly contacts an inner side of the wall.

The at least one through hole of the draining structure may form an elongated hole, a slot, a rectangular hole or a circular hole.

The draining structure may comprise a plurality of through holes, which are dispersed throughout the cradle, the wall, if present, and/or the cradle base, if present.

In an embodiment, the inner cross-sectional area of the at least one through hole of the draining structure is more than 0.01 mm² and less than 130 mm². In an embodiment the inner cross-sectional area of the at least one through hole of the draining structure is less than 65 mm², preferably less than 30 mm².

In an embodiment, the outer cradle side is hydrophobic. The outer cradle side may comprise a hydrophobic coating and/or is made from a hydrophobic material. In an embodiment, the cradle may be formed from a core material that is subsequently coated with a hydrophobic coating.

Presence of a hydrophobic material and the presence of a draining structure helps, in combination, that any volume or gap between the medical device and the cradle is vented to the environment, thereby ensuring that humidity and/or liquid such as water can evaporate and/or is transferred to the exterior, in particular to an area around the periphery of the cradle via the draining structure. The provision of a hydrophobic inner cradle side allows to work against capillary forces, which otherwise hinder removal of water, humidity and/or air out of a space between the proximal medical device side and the inner cradle side.

Optionally, further elements of the cradle, in particular a wall as explained before, may fully or partly have a hydrophobic coating. In particular, the inner cradle side may also have a hydrophobic coating. In an embodiment, the cradle is made from a hydrophobic material and/or the total surface of the cradle is coated with a hydrophobic coating.

Further, in some embodiments, the medical device, in particular the proximal medical device side, is hydrophobic, thereby further contributing to the effect of the hydrophobic inner cradle side. For this purpose, a proximal medical device side, such as a proximal housing wall of the medical device, or a housing of the medical device as a whole may be made from hydrophobic material and/or have a hydrophobic coating.

In an embodiment, the outer cradle side and the inner cradle side are substantially coplanar but may have structural elements such as ribs, protrusions, channels or recesses at either or both of the inner and outer cradle sides. In an embodiment, the cradle base is substantially plate-shaped. The outer cradle side may be curved, in particular concave, to contribute to a snug contact with a patient's skin surface.

The outer cradle side may comprise an adhesive layer for removably attaching the cradle to a patient's skin. The adhesive may cover the total outer cradle side or only one or more portions thereof. In a further embodiment, the cradle additionally or alternatively to an adhesive may comprise further skin attachment elements, such as a belt or a belt coupling structure.

In an embodiment, the cradle, in particular a cradle base, comprises a functional interface, the functional interface enables an operative coupling of the medical device with a patient's body, particularly, a patient's skin. Such functional interface may, for example, include a functional coupling aperture in the cradle through which a piercing element, such as an infusion cannula and/or an analyte sensor element may project beyond the outer cradle side and to pierce a patient's skin or get in contact with a patient's skin. A piercing element coupling structure may optionally be provided as part of the functional interface. The piercing element coupling structure is designed to engage the piercing element. In further embodiments, the cradle includes an integrated piercing element that projects from the outer cradle base side in the proximal direction.

In an embodiment, the cradle base and the circumferential wall at least partly delimit a continuous fluidic volume. The continuous fluidic volume is fluidically coupled to the draining structure. In case the medical device is coupled to the cradle, said fluidic volume is further delimited by the proximal medical device side. Such design ensures that humidity or liquid such as water that is present at a location between the cradle and the bases can be drained and evaporate to the exterior.

In an embodiment, the cradle includes spacers, the spacers projecting from the inner cradle side, preferably in a direction, which in use faces away from a patient's skin. Such spacers may, for example, be realized by posts that project from the cradle base. Such posts support the medical device housing at the proximal medical device side. Alternatively, or additionally, spacers may be realized by ribs that extend on the inner cradle side, thereby increasing the cradle stiffness. Such ribs may optionally have traverse venting openings in order to ensure fluidically coupling of the whole fluidic volume between the cradle and the medical device with the draining structure. The ribs thus help to prevent the forming of isolated fluidic compartments between the medical device and the cradle. In an embodiment, a continuous fluidic volume is present between the proximal medical device side and the inner cradle side in a configuration in which the medical device is coupled to the cradle. Alternatively, a number of compartments may be present between the medical device and the cradle, wherein each of the compartments are separately fluidically coupled with the exterior by a draining structure.

In an embodiment, the proximal medical device side and the inner cradle side are at least partly parallel to each other in a configuration in which the medical device and the cradle are coupled.

The medical device may have a medical device housing, such that a side of the medical device refers to an outer surface of the medical device housing. In particular, the expression “proximal medical device side” refers to an outer wall of the medical device, which in use is proximal to a patient's body.

In an embodiment, the cradle coupling structure and the medical device coupling structure are designed for releasable engagement, allowing the cradle and the medical device to be decoupled without damaging the medical device and/or the cradle. The medical device coupling structure of the cradle and the cradle coupling structure of the medical device may, in combination, for example, form a latch-catch arrangement and/or may form a snap-fit arrangement. Allowing a decoupling without damaging the medical device and in particular its cradle coupling structure may be required since the medical device may have a longer life time as compared to the cradle. The medical device thus can be reused with a number of cradles one after the other. The cradle and in particular its medical device coupling structure may or may not be damaged upon decoupling. In alternative embodiments, the cradle device coupling structure and the medical device coupling structure may be designed for a permanent coupling that cannot be decoupled or disengaged without destruction of the cradle device coupling.

In an embodiment, the medical device includes at least one of an infusion device and a continuous analyte measurement device. An infusion device may for example be an insulin infusion device that is designed to infuse insulin in a continuous or quasi-continuous manner according to a time-variable basal schedule and further infuse insulin boli on demand. A continuous analyte measurement device may for example be a continuous glucose meter that is designed for determining a glucose concentration e.g., in the blood or interstitial tissue by way of an electrochemical sensor.

According to a further aspect, the overall objective is achieved by a method for coupling a medical device with a patient's skin via a cradle. The method includes providing a medical system according to any embodiment as described above and/or further below. The method further includes coupling the cradle with a patient's skin. The method further includes coupling the medical device with the cradle by establishing an engagement between the medical device coupling structure and the cradle coupling structure.

Also disclosed is a method of venting a space between a medical device and a cradle. The method comprises the steps of providing a medical device and a cradle according to any embodiment as described above and/or below. The method further includes the step of engaging the cradle coupling structure of the medical device with the medical device coupling structure of the cradle, such that the proximal medical device side faces the inner cradle side. The method further comprises the step of venting a space between the medical device and the cradle by allowing removal of water, humidity or air of the space by means of the draining structure, wherein the draining structure is fluidically coupled to the space between the medical device and the cradle.

In an embodiment, the method of venting the space between the medical device and the cradle, the draining structure of the cradle comprises at least two through holes fluidically coupled to each other, wherein each of the at least two through holes fluidically couples the inner cradle side and the outer cradle side. The method may further include the step of blowing air or gas into one of the through holes from the outer cradle side thereby venting the draining structure by means of the provided flow of air or gas. Water and/or humidity is withdrawn from the draining structure by means of the flow of air or gas introduced into one of the at least through holes, and out another one of the at least one through holes.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1a shows an exemplary embodiment of a cradle in top view from distal toward proximal;

FIG. 1b shows the cradle of FIG. 1a in a perspective top view;

FIG. 1c shows the cradle of FIG. 1a in perspective bottom view;

FIG. 2a shows a medical system in accordance with the present disclosure in a schematic side view; and

FIG. 2b shows a medical system in accordance with the present disclosure in a schematic top view.

### DESCRIPTION

The embodiments described below are not intended to be exhaustive or to limit the invention to the precise forms disclosed in the following description. Rather, the embodiments are chosen and described so that others skilled in the art may appreciate and understand the principles and practices of this disclosure.

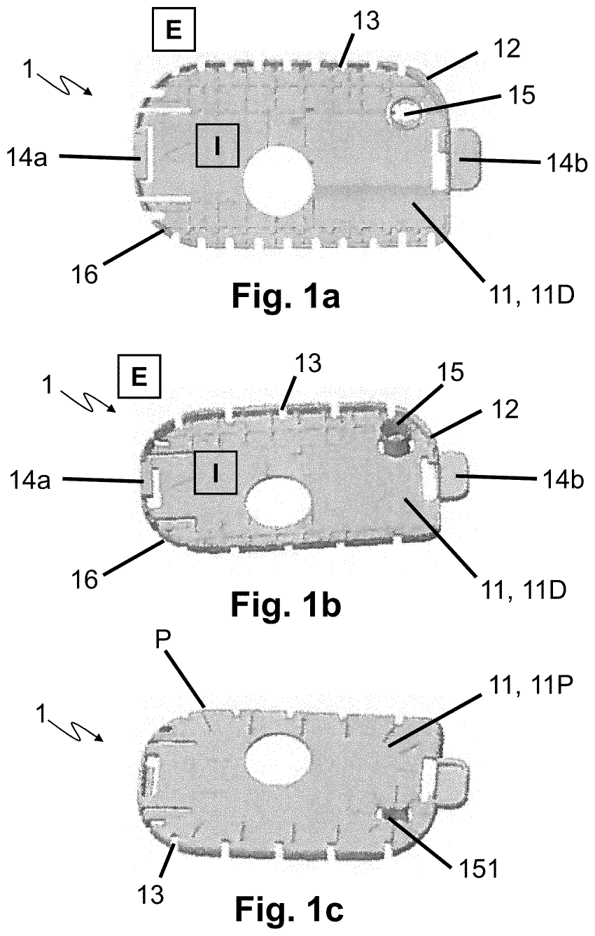

FIG. 1a, FIG. 1b, and FIG. 1c show an exemplary embodiment of a cradle 1 in accordance with the present disclosure in different views. FIG. 1a shows a top view of the inner cradle side, while FIG. 1b shows a perspective top view of the inner cradle side and FIG. 1c a perspective view of the outer cradle side.

In the shown embodiment, the cradle 1 is generally formed integrally from a single piece of plastic material by way of injection molding. The cradle 1 incudes a cradle base 11 having an outer cradle side 11P and an inner cradle side 11D. The outer cradle side 11P (best seen in FIG. 1c) is coated with a skin-compatible adhesive coating via which the cradle 1 is attachable respectively coupleable to a patient's skin. In use, i.e., in a state in which a medical device is coupled to the cradle, the inner cradle side 11D (best visible in FIG. 1a, 1b) faces the medical device.

A circumferential wall 12 projects from the cradle base 11 in a distal direction. The wall 12 comprises two opposing wall sides, wherein one of the two wall sides is at least partly formed by at least part of the outer cradle side 11P. The other of the two opposing walls sides is at least partly formed by at least part of the inner cradle side 11D. The circumferential wall 12 extends substantially along the whole cradle periphery 16. The area that is surrounded by the circumferential wall 12 is referred to as interior I and the outside area is referred to as exterior E, as indicated in FIG. 1a and FIG. 1b. The interior I defines the area where a medical device is arranged in a situation of use, such that the circumferential wall 12 circumferences the housing of the medical device at least in its proximal area.

A plurality of through holes 13 is arranged in the circumferential wall 12 such that the two opposing wall sides, which are at least partly formed by the at least part of the outer cradle side 11P and the at least part of the inner cradle side 11D, respectively, are fluidically coupled to each other by the draining structure or “drain,” which includes the through holes 13 that are formed as slits, which interrupt the wall 12 in a circumferential direction of the cradle 1. The through holes 13 extend from a free end of the wall 12 into the cradle base 11. Accordingly, the through holes 13 are not only located at the free end of the wall 12, but are also located at the transition of the wall 12 and the cradle base 11 (best visible in FIG. 1c). In this example, the draining openings 13 are distributed substantially equally along the circumferential wall 12. Other arrangements, however, may be used as well.

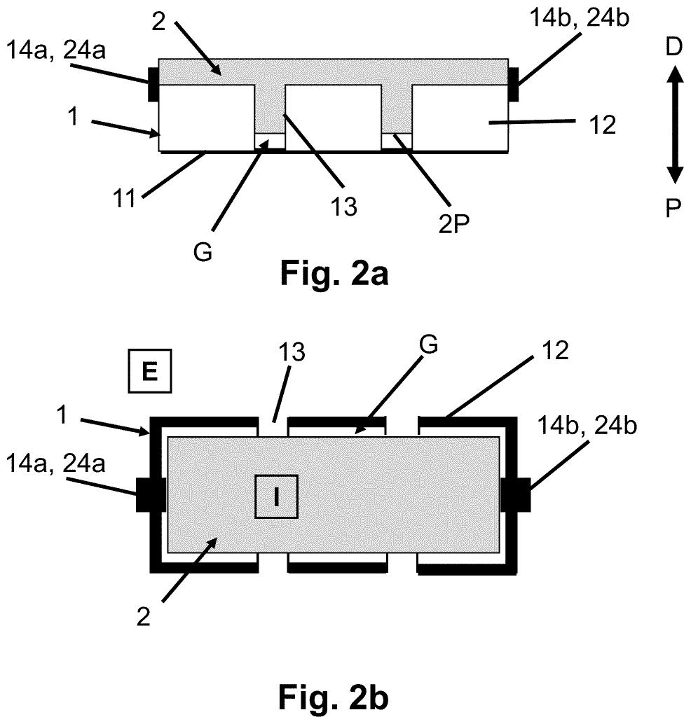

A medical device coupling structure (also referred to as a “medical device coupler”) 14a, 14b is formed integrally with the circumferential wall 12. The medical device coupling structure 14a, 14b includes elements at opposing sides of the cradle 1. When coupling the cradle 1 with a medical device, each of the elements of the medical device coupling structure 14a, 14b engages a corresponding counter-element of the cradle coupling structure (also referred to herein as a “cradler coupler”) 24a 24b that is provided at the medical device (as shown in FIGS. 2a, 2b). Element 14b is designed as a flap. By moving the flap 14b in the proximal direction, the engagement between the medical device coupling structure 14a, 14b, and the cradle device coupling structure 24a, 24b of the medical device can be released.

A functional interface 15 is provided in form of a tubular element that extends from the inner cradle side 11D towards the outer cradle side 11P, where it opens in a functional interface aperture 151 (best seen in FIG. 1a, FIG. 1c). The functional interface 15 is designed for receiving a skin piercing element, such as an infusion cannula and/or a transcutaneous sensor element.

The outer cradle side 11D further comprises a hydrophobic coating (not individually referenced). However, the inner side of the peripheral wall 12 may also comprise a hydrophobic coating. Other regions of the cradle 1 or its whole surface may optionally also be coated. In a variant, no coating is provided but the cradle 1 as a whole is formed from a hydrophobic material.

In the following, reference is additionally made to FIG. 2a and FIG. 2b. FIG. 2a shows a medical system, i.e., a kit of a cradle 1 and a medical device 2 in accordance with the present disclosure in a schematic side view, traverse to a proximal direction and distal direction, respectively. FIG. 2b shows a medical system 2 in a schematic top view of the inner cradle side 11D. The cradle 1 of the medical systems according to FIGS. 2a and 2b is similar to the cradle as shown in FIG. 1a to FIG. 1c.

The medical device 2 and the cradle 1 are releasably coupled via engagement of a medical device engagement structure 14a, 14b of the cradle 1 with complementary counterparts forming a cradle coupling structure 24a, 24b of the medical device 2.

In FIG. 2a, between the proximal side of the medical device 2 and the cradle base 11 respectively the inner cradle side (not individually referenced in FIG. 2a) there is a gap G. The gap G forms a hollow space, which is in fluidic communication with the exterior E via the draining structure. The through holes 13 forms a passage through which humidity, water and/or gas can flow from the inner cradle side 11D to the outer cradle side 11D. Accordingly, in use, i.e., in a state in which the medical device 2 is coupled to the cradle 1, the draining structure allows venting of the hollow space G without decoupling the medical device 2 and the cradle 1.

Similar in FIG. 2b, a gap G is present between the medical device 1 and the cradle 1. More precisely, the gap G is located between the medical device 1 and the inner cradle side 11D, which at least partly forms one wall side of the circumferential wall 12. Again, the through holes 13 fluidically couple the inner cradle side 11D with the outer cradle side 11P thereby allowing efficient ventilation of the space G.

It is noted that in both FIG. 2a and FIG. 2b the width of the gap G is significantly exaggerated for the sake of clarity.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF DESIGNATIONS

1 cradle
11 cradle base
11D inner cradle side
11P outer cradle side
12 circumferential wall
13 through holes of the draining structure
14a, 14b medical device coupling structure
15 functional interface
151 functional interface aperture
16 cradle periphery
2 medical device
24a, 24b cradle coupling structure
D distal direction
E exterior
I interior
G gap
P proximal direction

What is claimed is:

1. A cradle for a medical device, the cradle comprising:
an inner cradle side and an opposing outer cradle side, the outer cradle side being adapted for attachment to a patient's skin with at least part of the outer cradle side facing the patient's skin;
a medical device coupler adapted for coupling the medical device to the cradle such that part of the medical device faces the inner cradle side; and
a drain fluidly coupling the inner cradle side with the outer cradle side.

2. The cradle according to claim 1, wherein the drain comprises at least one through hole fluidly coupling the inner cradle side with the outer cradle side.

3. The cradle according to claim 2, further comprising:
a cradle base; and
a wall projecting away from the cradle base and having two opposing wall sides, one of the two opposing wall sides being at least partly formed by at least part of the outer cradle side and the other of the two opposing wall sides being at least partly formed by at least part of the inner cradle side, wherein the two opposing wall sides are fluidly coupled to each other by the drain.

4. The cradle according to claim 3, wherein the at least one through hole is located at a free end of the wall.

5. The cradle according to claim 3, wherein the at least one through hole forms an interruption of the wall.

6. The cradle according to claim 3, wherein the at least one through hole is located at the transition of the wall and the cradle base, thereby extending from the wall into the cradle base.

7. The cradle according to claim 3, wherein the wall forms a border strip that extends over at least half of the circumferential length of the cradle base.

8. The cradle according to claim 2, wherein the at least one through hole forms an elongated hole, a slot, a rectangular hole or a circular hole.

9. The cradle according to claim 2, wherein the cross-sectional area of the at least one through hole is more than 0.01 mm² and less than 5 mm².

10. The cradle according to claim 1, further comprising spacers projecting from the inner cradle side and configured for locating a medical device at a predefined distance from the inner cradle side.

11. The cradle according to claim 1, wherein the drain comprises at least one groove located on the inner cradle side.

12. The cradle according to claim 1, wherein the outer cradle side is hydrophobic.

13. A medical system, comprising:
a cradle according to claim 1;
a medical device having a cradle coupler complementary to the medical device coupler, the medical device having a proximal medical device side, wherein in a configuration in which the medical device is coupled to the cradle the proximal medical device side faces the inner cradle side.

14. The medical system according to claim 13, wherein, in a configuration in which the medical device is coupled to the cradle, the proximal medical device side and the inner cradle side delimits a hollow space fluidly coupled with the drain.

15. A method of venting a space between a medical device and a cradle, the method comprising:
providing a medical system according to claim 13;
engaging the cradle coupler with the medical device coupler, such that the proximal medical device side faces the inner cradle side; and
venting a space between the medical device and the cradle by allowing removal of water, humidity or air from the space via the drain, wherein the drain is fluidly coupled to the space between the medical device and the cradle.
